(19) United States
(12) Patent Application Publication
(10) Pub. No.: US 2020/0370094 A1
(43) Pub. Date: Nov. 26, 2020

(54) PRIMERS, KIT AND METHOD FOR DETECTING EHP PATHOGENS OF LITOPENAEUS VANNAMEI

(71) Applicant: Yellow Sea Fisheries Research Institute, Chinese Academy of Fishery Sciences, Qingdao (CN)

(72) Inventors: Xupeng LI, Qingdao (CN); Jie KONG, Qingdao (CN); Xianhong MENG, Qingdao (CN); Sheng LUAN, Qingdao (CN)

(21) Appl. No.: 16/878,791

(22) Filed: May 20, 2020

(30) Foreign Application Priority Data
May 21, 2019 (CN) 201910426172.8

(51) Int. Cl. C12Q 1/686; C12Q 1/6853

(52) U.S. Cl. CPC C12Q 1/686; C12Q 2600/16; C12Q 1/6853

(57) ABSTRACT

The present invention discloses primers, probes, a kit and a method for detecting Enterocytozoon hepatopenaei (EHP) pathogens of Litopenaeus vannamei. A PCR reaction system adopted in the method includes: a forward primer having a nucleotide sequence as shown in SEQ ID NO: 1, a reverse primer having a nucleotide sequence as shown in SEQ ID NO: 2, a forward primer having a nucleotide sequence as shown in SEQ ID NO: 4, a reverse primer having a nucleotide sequence as shown in SEQ ID NO: 5, a probe having a nucleotide sequence as shown in SEQ ID NO: 3, and a probe having a nucleotide sequence as shown in SEQ ID NO: 6. The method can perform duplex quantitative fluorescence PCR, an internal reference and a sample to be detected are put in the same reaction, thereby eliminating unstable factors of two reactions and improving the reliability of the result.

### TECHNICAL FIELD

The present invention relates to a method for detecting Enterocytozoon hepatopenaei (EHP) pathogens, and in particular to primers, a kit and a method for detecting EHP pathogens of Litopenaeus vannamei.

### BACKGROUND

Enterocytozoon hepatopenaei (EHP) is a highly infectious intracellular parasitic microsporidia, which mainly infects major shrimp breeding species including Litopenaeus vannamei. It is also one of the most important diseases in the shrimp breeding industry worldwide. EHP mainly infects a hepatopancreas tissue of a shrimp. After infecting the shrimp, the EHP produces mature spores in the hepatopancreas, which are discharged out of the body with feces, and then continue to infect other shrimps. The shrimps infected with EHP mainly show slow growth and uneven sizes, and the shrimps infected with EHP become susceptible to other pathogens, and the shrimps with severe diseases eventually die. Therefore, EHP infection brings huge economic losses to shrimp breeding industry.

The Litopenaeus vannamei is one of the most important shrimp breeding species in China and even in the world, and the species of Litopenaeus vannamei alone accounts for more than 80% of the domestic mariculture shrimp production. At present, EHP detection means for Litopenaeus vannamei include pathology, immunodiagnosis, probe hybridization, PCR technology, etc. Among them, the PCR technology is considered to be one of the best methods for EHP detection of Litopenaeus vannamei due to its features of simplicity, ease of operation and high accuracy. A conventional PCR technology can conduct qualitative detection of pathogens, while the quantitative fluorescence PCR technology can conduct quantitative detection of pathogens.

However, an ordinary quantitative fluorescence PCR needs to carry out two reactions on an internal reference and the pathogens separately. Such an operation, on one hand needs to take longer for an experiment; and on the other hand, may cause experimental errors due to the differences between two operations, which have a certain influence on the accuracy of the results.

### SUMMARY

An objective of the present invention is to provide primers, a kit and a method for detecting Enterocytozoon hepatopenaei (EHP) pathogens of Litopenaeus vannamei. The method solves the problem that the existing quantitative fluorescence PCR needs to carry out two reactions on an internal reference and the pathogens separately. In addition, the method can carry out duplex quantitative fluorescence PCR, puts the internal reference and a sample to be detected into the same reaction, removes unstable factors of the two reactions, and improves the reliability of the result.

In order to achieve the aforementioned objective, the present invention provides a primer for amplifying an 18S rRNA gene of Litopenaeus vannamei, which includes a forward primer having a nucleotide sequence as shown in SEQ ID NO: 1 and a reverse primer having a nucleotide sequence as shown in SEQ ID NO: 2.

The present invention further provides a fluorescent probe, which is used against the primer for amplifying an 18S rRNA gene of Litopenaeus vannamei and has a nucleotide sequence as shown in SEQ ID NO: 3.

Preferably, the probe is labeled with a VIC fluorophore and a BHQ1 quencher group. The present invention further provides a primer for amplifying a SWP gene of EHP pathogens of Litopenaeus vannamei, which includes a forward primer having a nucleotide sequence as shown in SEQ ID NO: 4 and a reverse primer having a nucleotide sequence as shown in SEQ ID NO: 5.

The present invention further provides a fluorescent probe, which is used against the primer for amplifying a SWP gene of EHP pathogens of Litopenaeus vannamei and has a nucleotide sequence as shown in SEQ ID NO: 6.

Preferably, the probe is labeled with a FAM fluorophore and a BHQ1 quencher group.

The present invention further provides a kit for quantitatively detecting EHP pathogens of Litopenaeus vannamei, which includes a forward primer having a nucleotide sequence as shown in SEQ ID NO: 1, a reverse primer having a nucleotide sequence as shown in SEQ ID NO: 2, a forward primer having a nucleotide sequence as shown in SEQ ID NO: 4, a reverse primer having a nucleotide sequence as shown in SEQ ID NO: 5, a probe having a nucleotide sequence as shown in SEQ ID NO: 3, and a probe having a nucleotide sequence as shown in SEQ ID NO: 6.

Preferably, the kit further includes: Premix Ex Taq (Probe qPCR) (2×), and ROX Reference Dye (50×).

The present invention further provides a method for quantitatively detecting EHP pathogens of Litopenaeus vannamei, which includes the following steps:

taking the extracted tissue DNA of Litopenaeus vannamei as a sample to be detected;

placing a PCR reaction system into a PCR reaction tube, placing the PCR reaction tube into a quantitative fluorescence PCR instrument, setting a reaction time, and carrying out a duplex PCR reaction.

The PCR reaction system includes a forward primer having a nucleotide sequence as shown in SEQ ID NO: 1, a reverse primer having a nucleotide sequence as shown in SEQ ID NO: 2, a forward primer having a nucleotide sequence as shown in SEQ ID NO: 4, a reverse primer having a nucleotide sequence as shown in SEQ ID NO: 5, a probe having a nucleotide sequence as shown in SEQ ID NO: 3, a probe having a nucleotide sequence as shown in SEQ ID NO: 6, Premix Ex Taq (Probe qPCR) (2×), ROX Reference Dye (50×), and a sample.

The sample is a sample to be detected, a standard, a negative control or a blank control. The standard includes plasmids containing an 18S rRNA gene of Litopenaeus vannamei at a known concentration and plasmids containing an EHP SWP gene at a known concentration, and meanwhile the standard is used as a positive control. The negative control includes DNAs extracted from tissues of healthy Litopenaeus vannamei. The blank control includes H₂O.

The 18S rRNA gene of Litopenaeus vannamei serves as the internal reference.

When both the positive control and the internal reference have amplification product, the blank control has no amplification product, and the negative control only has amplification of the 18S rRNA gene product of Litopenaeus vannamei and has no amplification of the EHP SWP gene product, it indicates that the PCR reaction is normal. If the sample to be detected has amplification of the EHP SWP gene product, it indicates that the detection result is positive; and if the sample to be detected has no amplification of the EHP SWP gene product, it indicates that the detection result is negative. Linear regression is conducted according to a standard curve and Ct values corresponding to the product to obtain the EHP pathogen content in the sample to be detected.

Preferably, the extraction of the tissue DNAs of Litopenaeus vannamei includes: taking the Litopenaeus vannamei to be detected, taking out hepatopancreas tissues, adding a lysis buffer, fully grinding the hepatopancreas tissues, adding a proteinase K, mixing evenly, then placing in a water bath, and adding phenol to obtain the tissue DNAs of Litopenaeus vannamei.

The primers, kit and method for detecting the EHP pathogens of Litopenaeus vannamei of the present invention solve the problem that the existing quantitative fluorescence PCR needs to carry out two reactions on the internal reference and the pathogens separately, and have the following advantages:

(1) the method of the present invention avoids the situation that the conventional quantitative fluorescence PCR method divides the internal reference and the pathogens to be detected into two reactions, puts the internal reference and the sample to be detected into the same reaction, removes the unstable factors of the two reactions, and improves the reliability of the result of the comparison between the sample to be detected and the internal reference;

(2) the method of the present invention avoids the situation that the conventional quantitative fluorescence PCR method divides the internal reference and the sample to be detected into two reactions, can complete the internal reference and the sample to be detected in one reaction, and thus shortens the original experiment period by half;

(3) the method of the present invention has high sensitivity, and the detectable minimum concentration of a positive sample is 1×10² copies/μL; and

(4) the method of the present invention has strong anti-pollution capability. Compared with a conventional PCR, in the present invention, it does not need to open the PCR reaction tube for subsequent operation after PCR amplification, so as to avoid the pollution caused by leakage of positive products after the PCR amplification.

### BRIEF DESCRIPTION OF THE DRAWING

FIG. 1 is a standard curve prepared according to the PCR amplification result of the standard;

FIG. 2 is a duplex PCR amplification graph; and

FIG. 3 shows amplification curves corresponding to a standard (positive control), a negative control, a blank control, a positive sample and a negative sample.

### DETAILED DESCRIPTION

The following clearly and completely describes the technical solutions in the embodiments of the present invention. Apparently, the described embodiments are merely some embodiments of the present invention rather than all of the embodiments. All other embodiments obtained by a person of ordinary skill in the art based on the embodiments of the present invention without creative efforts shall fall within the protection scope of the present invention.

Embodiment 1 Experiment for EHP detection of Litopenaeus vannamei

(1) DNA Extraction

A lysis buffer was prepared and its components were: 100 mM of Tris-HCl (pH 8.0), 50 mM of EDTA, and 0.5% of SDS (sodium dodecyl sulfate).

The hepatopancreas of Litopenaeus vannamei to be detected was taken, grinded, weighed at 100 mg, added with 400 μL of the aforementioned lysis buffer to fully grind the hepatopancreas tissues, added with 80 μg of a proteinase K, mixed fully, then placed in a water bath at 55° C. for 1 h, added with 400 μL of phenol, gently shaken for 10 min, and centrifuged at 12000 r/min for 10 min. The supernatant was taken, added with 400 μL of phenol-chloroform (1:1) and mixed uniformly, and centrifuged at 12000 r/min for 10 min. The supernatant was taken, added with 400 μL of chloroform and mixed uniformly, and centrifuged at 12000 r/min for 10 min. The supernatant was taken, added with absolute ethanol precooled at −20° C. which was twice the volume of the supernatant, and mixed uniformly, and centrifuged at 12000 r/min for 10 min. The sediment was retained, washed twice with 400 μL of ethanol that had a concentration of 75% and precooled at −20° C., air-dried at room temperature, and then added with 50 μL of ultrapure water to obtain extracted DNAs.

(2) Duplex Quantitative Fluorescence PCR

Plasmids containing part of the 18S rRNA gene of Litopenaeus vannamei and plasmids containing part of the Enterocytozoon hepatopenaei (EHP) SWP gene were uniformly diluted to a concentration of 1×10⁸ copies/μL. The two plasmids were mixed and diluted with gradients to concentrations of 1×10⁷ copies/μL, 1×10⁶ copies/μL, and 1×10⁵ copies/μL, so as to serve as standards, and also positive controls.

A quantitative fluorescence PCR reaction system was formulated, which included: 10 μL of Premix Ex Taq (Probe qPCR) (2×), 0.4 μL of primer 18S rRNA-F (10 μM), 0.4 μL of primer 18S rRNA-R (10 μM), 0.4 μL of primer EHP-F (10 μM), 0.4 μL of primer EHP-R (10 μM), 0.4 μL of probe 18S rRNA-probe (10 μM), 0.4 μL of probe EHP-probe (10 μM), 0.2 μL of ROX Reference Dye (50×), 1 μL of DNAs of the sample to be detected (200 ng/μL), or 1 μL of the aforementioned mixed plasmids (as standards and positive controls) or 1 μL of the DNAs extracted from tissues of healthy Litopenaeus vannamei (200 ng/μL) (as negative controls) or 1 μL of H₂O (as blank controls), with the ultrapure water making up to 20 pt. Premix Ex Taq (Probe qPCR) (2×) and ROX Reference Dye (50×) were purchased from Takara.

The nucleotide sequences of respective primers and probes were as follows.

The nucleotide sequence of the primer 18S rRNA-F (forward primer I) was:

(SEQ ID NO: 1)
AGCAGGCTGGTTTTTGCTTA;

The nucleotide sequence of the primer 18S rRNA-R (reverse primer I) was:

(SEQ ID NO: 2)
GTTCCGAAAAACCGACAAAA;

The nucleotide sequence of the probe 18S rRNA-probe (probe I) was:

(SEQ ID NO: 3)
CCCGAATGGTCGTGCATGGA;

The nucleotide sequence of the primer EHP-F (forward primer II) was:

(SEQ ID NO: 4)
GCGGCACAATTCTCAAACAT;

The nucleotide sequence of the primer EHP-R (reverse primer II) was:

(SEQ ID NO: 5)
TCCTACAAATGCTGTGTCTGTG;

The nucleotide sequence of the probe EHP-probe (probe II) was:

(SEQ ID NO: 6)
TCACCATTGGTCAAATACAATTTCAAACA.

The aforementioned probe 18S rRNA-probe was labeled with a VIC fluorophore and a BHQ1 quencher group, and the labeled probe was:

(SEQ ID NO: 3)
VIC-CCCGAATGGTCGTGCATGGA-BHQ1.

The aforementioned probe EHP-probe was labeled with a FAM fluorophore and a BHQ1 quencher group, and the labeled probe was:

(SEQ ID NO: 6)
FAM-TCACCATTGGTCAAATACAATTTCAAACA-BHQ1.

The quantitative fluorescence PCR instrument of ABI 7500 was used, and the reaction program was set as follows: pre-denaturation at 95° C. for 30 s, one cycle; 95° C. for 5 s, 60° C. for 34 s, 40 cycles.

After the quantitative fluorescence PCR reaction was completed, both the positive controls (mixed plasmids) and the internal reference (the 18S rRNA gene of Litopenaeus vannamei from the sample to be detected) had PCR amplification products, the blank controls (H₂O) had no PCR amplification product, and the negative controls (DNAs extracted from the tissues of healthy Litopenaeus vannamei) only had PCR amplification products of the 18S rRNA gene of Litopenaeus vannamei and had no PCR amplification products of the EHP SWP gene, which could prove that the experimental operation was correct.

The 18S rRNA gene fragment sequence of the amplified standards and positive controls (SEQ ID NO: 7) was:

AGCAGGCTGGTTTTTGCTTACAGCCCGAATGGTCGTGCATGGAATGATGG
AACAGGACCTCGGTTCTATTTTGTCGGTTTTTCGGAAC.

The EHP SWP gene fragment sequence of the amplified standards and positive controls (SEQ ID NO: 8) was:

GCGGCACAATTCTCAAACATTTTCACCATTGGTCAAATACAATTTCAAAC
ACTGTAAACCTTAAAGCATTAAAAAGAGACGATATTTACACAGACACAGC
ATTTGTAGGA.

The sample to be detected had amplification products of the EHP SWP gene, indicating that the detection result was positive. The content of EHP pathogens in the sample to be detected could be obtained by conducting linear regression according to the standard curve and the Ct values corresponding to the products (the number of cycles as experienced when the fluorescence signal in each reaction tube reached a set threshold value).

There were no amplification products of the EHP SWP gene for the sample to be detected, which indicated that the detection result was negative.

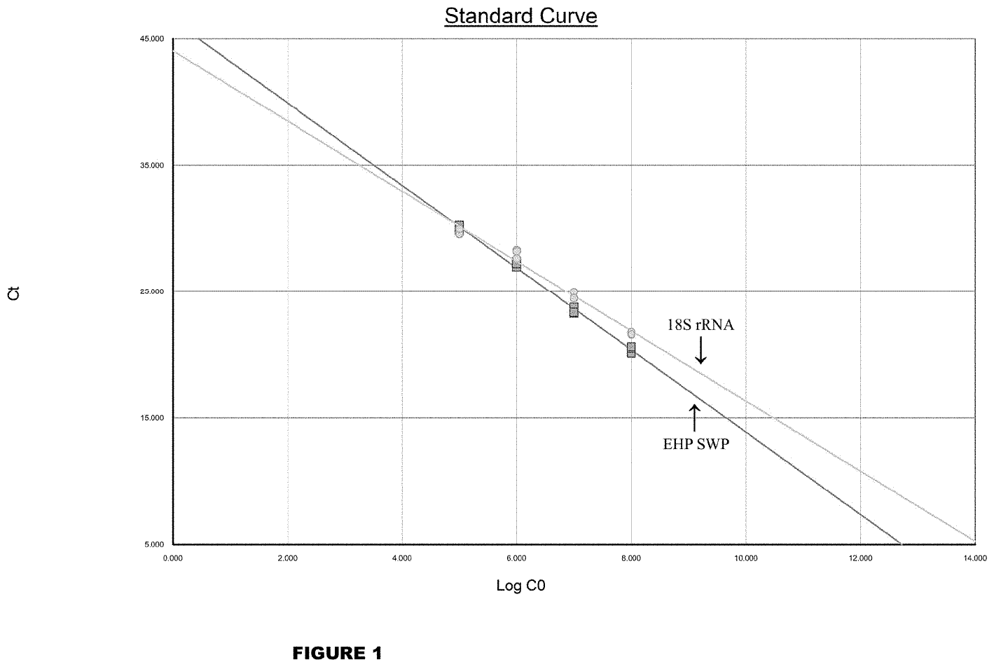

As shown in FIG. 1, it was a standard curve prepared according to the PCR amplification result of the standards (the upper curve was the standard curve corresponding to the 18S rRNA gene of Litopenaeus vannamei, the lower curve was the standard curve corresponding to the EHP SWP gene, and CO was the concentration of the standards).

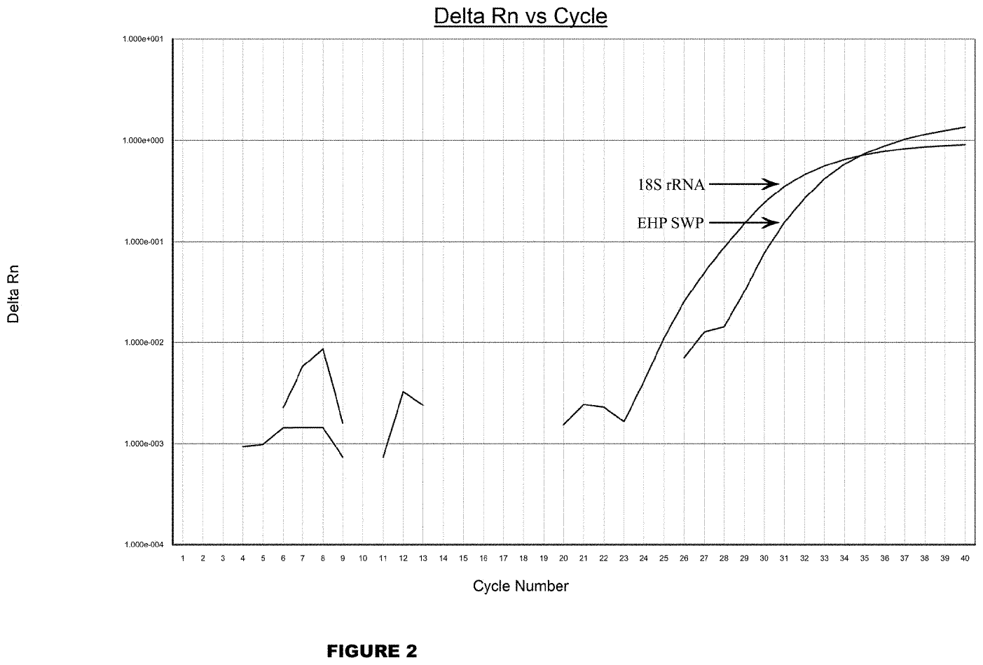

As shown in FIG. 2, it was a duplex PCR amplification graph of the same sample (in the figure, the upper peak was the amplification curve of the 18S rRNA gene of Litopenaeus vannamei (as the internal reference); the lower peak was the amplification curve (positive) of the EHP SWP gene, and Rn was the intensity of fluorescence signal).

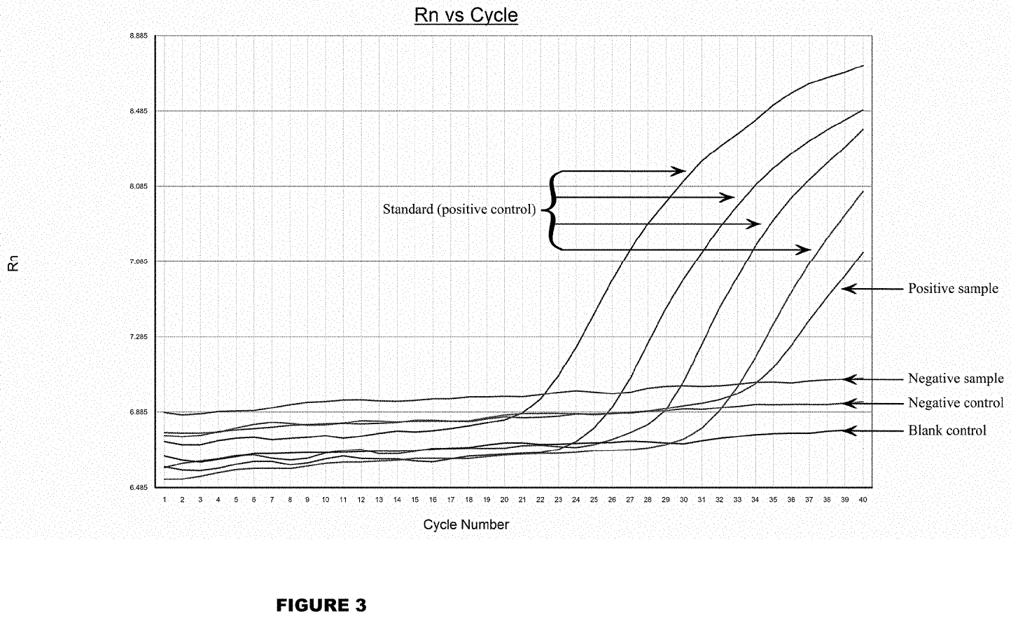

As shown in FIG. 3, it showed the amplification curves corresponding to the standards (i.e., positive controls), negative controls, blank controls, positive samples and negative samples, where the detection result of the positive samples was positive and the detection result of the negative samples was negative.

The method of the present invention has higher sensitivity, can detect the positive samples at a minimum concentration of 1×10² copies/μL, and shortens the original experiment period by half.

Although the content of the present invention has been described in detail through the aforementioned preferred embodiments, it should be recognized that the above description should not be considered as limiting the present invention. Upon reading the aforementioned content, it will be apparent to those skilled in the art that various modifications and alternations to the present invention can be made. Therefore, the claimed scope of the present invention shall be defined by the appended claims.

What is claimed is:

1. A primer for amplifying an 18S rRNA gene of Litopenaeus vannamei, comprising a forward primer having a nucleotide sequence as shown in SEQ ID NO: 1 and a reverse primer having a nucleotide sequence as shown in SEQ ID NO: 2.

2. A fluorescent probe, which is used against the primer for amplifying an 18S rRNA gene of Litopenaeus vannamei according to claim 1 and has a nucleotide sequence as shown in SEQ ID NO: 3.

3. The fluorescent probe according to claim 2, wherein the probe is labeled with a VIC fluorophore and a BHQ1 quencher group.

4. A primer for amplifying a gene of Enterocytozoon hepatopenaei (EHP) pathogens of Litopenaeus vannamei, which comprises a forward primer having a nucleotide sequence as shown in SEQ ID NO: 4 and a reverse primer having a nucleotide sequence as shown in SEQ ID NO: 5.

5. A fluorescent probe, which is used against the primer for amplifying a gene of EHP pathogens of Litopenaeus vannamei according to claim 4 and has a nucleotide sequence as shown in SEQ ID NO: 6.

6. The fluorescent probe according to claim 5, wherein the probe is labeled with a FAM fluorophore and a BHQ1 quencher group.

7. A kit for quantitatively detecting EHP pathogens of Litopenaeus vannamei, which comprises a forward primer having a nucleotide sequence as shown in SEQ ID NO: 1, a reverse primer having a nucleotide sequence as shown in SEQ ID NO: 2, a forward primer having a nucleotide sequence as shown in SEQ ID NO: 4, a reverse primer having a nucleotide sequence as shown in SEQ ID NO: 5, a probe having a nucleotide sequence as shown in SEQ ID NO: 3, and a probe having a nucleotide sequence as shown in SEQ ID NO: 6.

8. The kit for quantitatively detecting EHP pathogens of Litopenaeus vannamei according to claim 7, further comprising Premix Ex Taq (Probe qPCR) (2×) and ROX Reference Dye (50×).

9. A method for quantitatively detecting EHP pathogens of Litopenaeus vannamei, comprising:
taking the extracted tissue DNA of Litopenaeus vannamei as a sample to be detected; and
placing a PCR reaction system into a PCR reaction tube, placing the PCR reaction tube into a quantitative fluorescence PCR instrument, setting a reaction time, and carrying out a duplex PCR reaction;
wherein the PCR reaction system comprises a forward primer having a nucleotide sequence as shown in SEQ ID NO: 1, a reverse primer having a nucleotide sequence as shown in SEQ ID NO: 2, a forward primer having a nucleotide sequence as shown in SEQ ID NO: 4, a reverse primer having a nucleotide sequence as shown in SEQ ID NO: 5, a probe having a nucleotide sequence as shown in SEQ ID NO: 3, a probe having a nucleotide sequence as shown in SEQ ID NO: 6, Premix Ex Taq (Probe qPCR) (2×), ROX Reference Dye (50×), and a sample;
the sample is a sample to be detected, a standard, a negative control or a blank control; the standard comprises plasmids containing an 18S rRNA gene of Litopenaeus vannamei at a known concentration and plasmids containing a EHP SWP gene at a known concentration, and meanwhile the standard is used as a positive control; the negative control comprises DNAs extracted from tissues of healthy Litopenaeus vannamei; and the blank control comprises H₂O;
wherein the 18S rRNA gene of Litopenaeus vannamei is used as an internal reference;
when both the positive control and the internal reference have amplification product, the blank control has no amplification product, and the negative control only has amplification of the 18S rRNA gene product of Litopenaeus vannamei and has no amplification of the EHP SWP gene product, it indicates that the PCR reaction is normal; if the sample to be detected has amplification of the EHP SWP gene product, it indicates that the detection result is positive; and
if the sample to be detected has no amplification of the EHP SWP gene product, it indicates that the detection result is negative; and
linear regression is conducted according to a standard curve and Ct values corresponding to the product to obtain the EHP pathogen content in the sample to be detected.

10. The method for quantitatively detecting EHP pathogens of Litopenaeus vannamei according to claim 9, wherein the extraction of the tissue DNAs of Litopenaeus vannamei comprises: taking the Litopenaeus vannamei to be detected, taking out hepatopancreas tissues, adding a lysis buffer, fully grinding the hepatopancreas tissues, adding a proteinase K, mixing evenly, then placing in a water bath, and adding phenol to obtain the tissue DNAs of Litopenaeus vannamei.


SEQUENCE LISTING (8 sequences)

SEQ ID NO: 1 - DNA, 20 bp, artificial sequence
  The nucleotide sequence of the primer 18S rRNA-F
agcaggctggtttttgctta

SEQ ID NO: 2 - DNA, 20 bp, artificial sequence
  The nucleotide sequence of the primer 18S rRNA-R
gttccgaaaaaccgacaaaa

SEQ ID NO: 3 - DNA, 20 bp, artificial sequence
  The nucleotide sequence of the probe 18S rRNA-probe
cccgaatggtcgtgcatgga

SEQ ID NO: 4 - DNA, 20 bp, artificial sequence
  The nucleotide sequence of the primer EHP-F
gcggcacaattctcaaacat

SEQ ID NO: 5 - DNA, 22 bp, artificial sequence
  The nucleotide sequence of the primer EHP-R
tcctacaaatgctgtgtctgtg

SEQ ID NO: 6 - DNA, 29 bp, artificial sequence
  The nucleotide sequence of the probe EHP-probe
tcaccattggtcaaatacaatttcaaaca

SEQ ID NO: 7 - DNA, 88 bp, artificial sequence
  The 18S rRNA gene fragment sequence of the amplified standards and positive controls
agcaggctggtttttgcttacagcccgaatggtcgtgcatggaatgatggaacaggacctcggttctattttgtcggtttttcggaac

SEQ ID NO: 8 - DNA, 110 bp, artificial sequence
  The EHP SWP gene fragment sequence of the amplified standards and positive controls
gcggcacaattctcaaacattttcaccattggtcaaatacaatttcaaacactgtaaaccttaaagcattaaaaagagacgatatttacacagacacagcatttgtagga